(19) United States
(12) Patent Application Publication
(10) Pub. No.: US 2005/0113936 A1
(43) Pub. Date: May 26, 2005

(54) Surface treatments and modifications using nanostructure materials

(76) Inventors: John R. Brustad, Dana Point, CA (US); Nabil Hilal, Laguna Niguel, CA (US); Gary M. Johnson, Mission Viejo, CA (US); Charles C. Hart, Summerville, SC (US)

Correspondence Address: APPLIED MEDICAL RESOUCES CORPORATION, 22872 Avenida Empresa, Rancho Santa Margarita, CA, 92688, US

(21) Appl. No.: 10/976,506

(22) Filed: Oct. 29, 2004

Related U.S. Application Data
(60) Provisional application No. 60/516,197, filed on Oct. 30, 2003.

(51) Int. Cl. A61F 2/04; B05D 3/00; A61F 2/02

(52) U.S. Cl. 623/23.71; 427/2.1; 623/1.46; 623/23.74

(57) ABSTRACT

The invention is directed to nanostructure surface treatments, coatings or modifications formed from nanoscale building blocks. The nanostructure surface treatments, modifications or coatings have hydrophobic, hydrophilic and surface adherence properties. The nanoscale building blocks have orientation, geometry, packing density and composition that may be adjusted to control the unique surface characteristics of the desired treatment, coating or modification. Applications of this nanostructure technology include surgical clips, staples, retractors, sutures and manipulators where an improvement in traction, retention or occlusion is desired without excessive material or tissue deformation or where high compressive forces would be undesirable, dangerous or ineffective. In one aspect, a nanostructure surface treatment for a medical device having an external surface is disclosed, wherein the treatment is applied on the external surface to provide a hydrophobic or a hydrophilic surface. With this aspect, the treatment comprises titanium dioxide and provides nanoscopic structures having nearly vertical sidewalls. The treated surface of the device has contact angles greater than or equal to 150 degrees. The vertical sidewalls provide a negative capillary effect and have a width of about 200 nm. The vertical sidewalls attach to a wet surface by the negative capillary effect. The van der Waals forces of the vertical sidewalls enable the treated surface to attach to a dry surface. The treatment may be vapor deposited and cured on the device, or the treatment may be laser blasted on the device.

This is a non-provisional application claiming the priority of provisional application Ser. No. 60/516,197, filed on Oct. 30, 2003, entitled “Nanostructure Surface Treatments,” which is fully incorporated herein by reference.

### BACKGROUND OF THE INVENTION

1. Field of the Invention

This invention generally relates to surface treatments and, in particular, to surface treatments, modifications or coatings using nanostructure materials having hydrophobic, hydrophilic, germicidal or lubricious properties.

2. Discussion of the Prior Art

Coatings are commonly used for a variety of applications. Paint is often used to provide environmental protection. Oil is used to provide lubrication between moving parts. Powders of various sorts may be used to maintain dryness and to lubricate. Waxes may be used to repel water. The advantages of appropriate surface coatings or modifications are well understood and appreciated. However, many of the coatings of the prior art fall short of their intended use due to the physical bond between the coating and the material that is coated.

Coatings, surface treatments or surface modifications using nanomaterials produce effects that are more effective and longer lasting than traditional coatings. For example, metallic stainless steel coatings sprayed with nano-crystalline powders demonstrate increased hardness when compared to conventional coatings. Plasma or thermal sprays may be applied to a surface to form a thin, hard ceramic nanocoating. These coatings may be made with titanium dioxide and a plasma torch, and sprayed onto metal surfaces. Such an application renders metals very resistant to corrosion. A unique value of nanoparticles is their extremely high particle surface area. This feature means that there are many more sites for achieving property enhancements.

Nanotechnology is a broad and interdisciplinary area of research and development that has potential for revolutionizing the ways in which materials and products are created and the range and nature of functionalities that can be accessed. In particular, the synthesis and control of materials in nanometer dimensions can access new material properties and device characteristics in unprecedented ways, and work is rapidly expanding worldwide in exploiting the opportunities offered through nanostructuring. More specifically, there is currently a need in the medical device art to incorporate nanostructuring to provide, among other things, thin film coatings having stronger bonds and better flexibility.

### SUMMARY OF THE INVENTION

The present invention is directed to nanostructure surface treatments, coatings or modifications formed from nanoscale building blocks. The nanostructure surface treatments, modifications or coatings have hydrophobic, hydrophilic and surface adherence properties. The nanoscale building blocks have orientation, geometry, packing density and composition that may be adjusted to control the unique surface characteristics of the desired treatment, coating or modification. Applications of this nanostructure technology include surgical clips, staples, retractors, sutures and manipulators where an improvement in traction, retention or occlusion is desired. In one aspect, the tissue contacting surfaces of a clip or retractor may be treated or coated with a nanostructure comprising a microscopically rough surface. In another aspect, polypropylene may be dissolved in a solvent, which may then be exposed to a precipitating agent and subsequently applied to an instrument surface. Next, the solvent mixture is evaporated in a vacuum oven. This results in a highly porous gel coating having a contact angle of at least 150 degrees.

Reusable instruments that must be sterilized before reuse may profit from nanoscale surface technology to render them more easily and effectively cleaned and more durable. Electrosurgical devices that normally become fouled with burned tissue during use may profit from nanoscale surface technology where the surfaces remain free from contamination and therefore continue effective. In addition, there are many devices that may also benefit from nanoscale surface technology such as catheters, access tubes, stents and grafts. For example, each of these devices may be treated with nanoscale surface technology so as to have specific characteristics on one surface and different characteristics on another surface. That is, a stent or graft may be treated to have a hydrophobic exterior and a hydrophilic interior, or vice versa. An access tube for use in the vascular system or urinary tract may be treated to enhance placement by having the exterior surface nanocoated with a lubricious coating while having an interior surface treated to inhibit clotting or encrustation. In this case, the external surface nanostructure may comprise a surface of hydrophobic material that is profiled with microscopic structures having nearly vertical sidewalls. Water becomes supported by the tips of the structures due to negative capillary effect. Each water droplet has a very high contact angle and a low sliding resistance on such a surface. However, if an external pressure exceeds the negative capillary pressure, the surface becomes wetted and is not water repellant any longer. The pressure is that exerted upon the surface by the walls of the vessel or duct into which it is being inserted or placed. Until that pressure is achieved, the surface remains hydrophobic.

The lumen of the access tube, on the other hand, may be treated or coated with a flouroalkylsilane so that the silane is anchored to the internal surface through conventional hydrolysis and condensation reactions. This coating results in reduced surface tension. Such a nanostructure may be applied by existing processes template, screen printing, electrostatic glazing or spraying. A stent or graft may have fibers that are treated with nanostructure technology to promote or inhibit ingrowth. Infection may be inhibited in the case of implanted or indwelling devices by the application of nanoscale materials to modify, coat or treat surfaces that are in contact with tissue or body product. In addition, nanomaterials may provide an opportunity to relieve the stress placed upon an immune system by the introduction of a foreign body. For instance, a heart valve, bladder valve, stent, graft, artificial bladder, transplanted kidneys or hearts or mechanical joints may all be treated with nanomaterials that render them invisible to the immune system and therefore un-rejected by an immune system.

Nanostructure materials may also be applied to surfaces that must conduct delicate or sensitive components such as blood. For example, an anastomosis where two or more vessels are connected may benefit from treated suture that does not promote the formation of clot. A heart valve could be treated with nanostructured components that prevent or reduce turbulence in the blood flow. Skin grafts or tissue grafts may benefit from properties that derive from application of nanostructured materials. This may be especially true of artificial skin or cultured skin to be used in the treatment of burn victims. In this case, one side of the graft may be treated with a nanomaterial that promotes tissue generation while the opposite side is treated with anti-microbial agents or other desirable components.

These and other features of the invention will become more apparent with a discussion of the various embodiments in reference to the associated drawings.

### DESCRIPTION OF THE DRAWINGS

The accompanying drawings, which are included in and constitute a part of this specification, illustrate the embodiments of the invention and, together with the description, explain the features and principles of the invention.

FIGS. 1 and 2 illustrate the comparative sizes of nanoscale measurements;

FIGS. 3A-3C illustrate the contact angles of water on smooth glass, on glass treated with Teflon (PTFE), and on glass treated with a superhydrophobic nanostructure material, respectively;

FIG. 4 illustrates a device or surface that is coated with a nanostructure;

FIG. 5 illustrates a tissue surface that is contacted by a nanocoating;

FIGS. 6A and 6B illustrate a surgical clip in a non-coated and nanocoated condition, respectively;

FIG. 7 illustrates a staple that is uncoated;

FIG. 8 illustrates a staple that is nanocoated in accordance with the invention;

FIG. 9 is an end view of a vessel graft having a nanocoating or nanostructure in accordance with the invention;

FIG. 10 is an end view of a vessel stent having a nanocoating or nanostructure in accordance with the invention;

FIG. 11 is a perspective view of a vessel fitted with a stent or graft in accordance with the invention;

FIG. 12 is an end view of a length of monofilament suture in accordance with the invention;

FIG. 13 is an end view of a length of stranded suture in accordance with the invention;

FIG. 14 illustrates suture placement in accordance with the invention;

FIG. 15 shows an electrosurgical device that is uncoated;

FIG. 16 shows an electrosurgical device that is nanocoated in accordance with the invention;

FIG. 17 is a perspective illustration of an artificial urinary bladder having nanocoatings in accordance with the invention;

FIG. 18 is a section view of an artificial urinary bladder having nanocoatings in accordance with the invention;

FIG. 19 is a perspective view of a fabric or mesh that is nanocoated or made of nanocoated fibers in accordance with the invention;

FIG. 20 is a perspective view of a nanocoated scissor in accordance with the invention;

FIG. 21 is a side view of an anastomosis device in accordance with the invention; and

FIG. 22 is a side view of a dialysis port in accordance with the invention.

### DESCRIPTION OF THE INVENTION

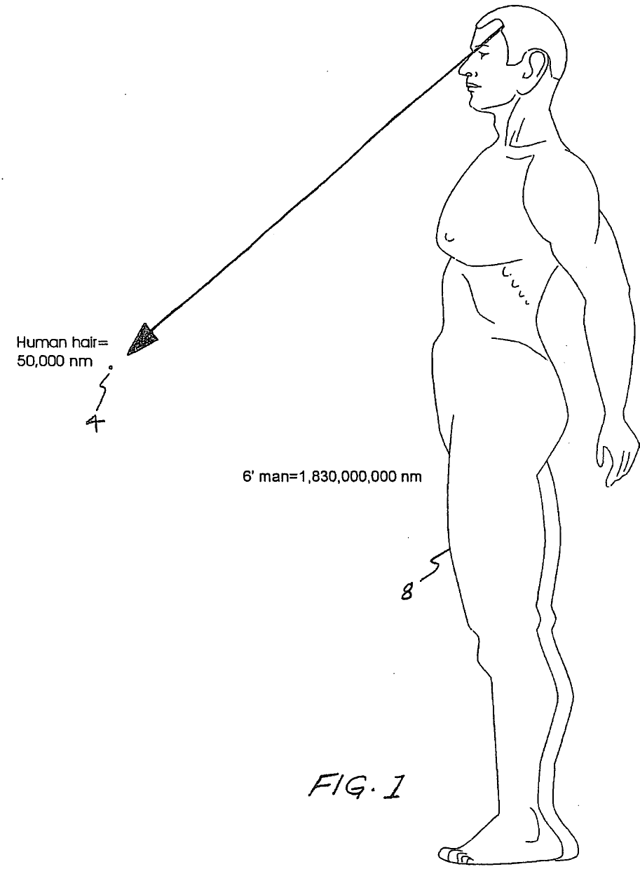

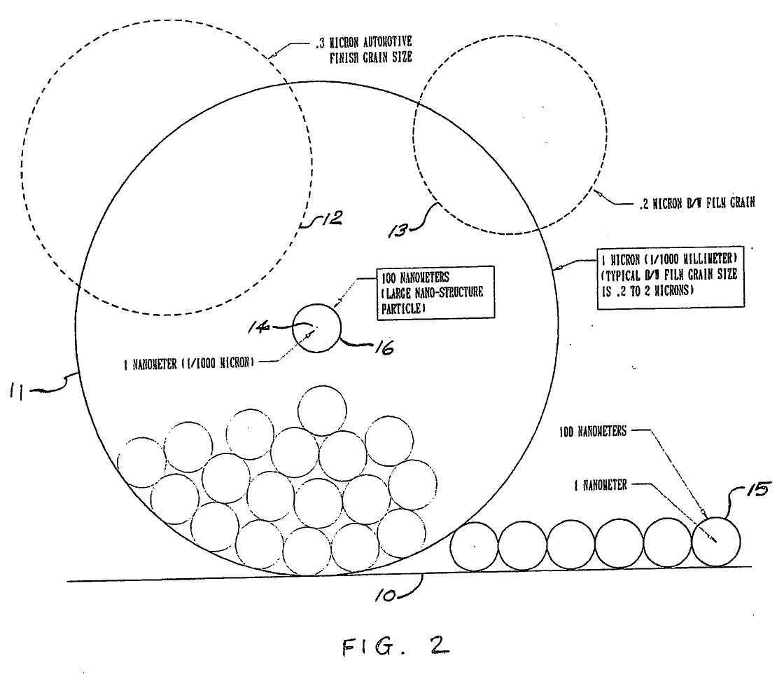

Referring to FIG. 1, there are shown comparable measurements of a human hair 4 and a six-foot tall man 8 shown in nanometer. A nanometer is one-billionth of a meter. The six-foot tall man 8 is approximately 1,830,000,000 nanometers tall. A normal human hair 4 is approximately 50,000 nanometers in diameter. A further comparison can be seen in FIG. 2, where a particle 11 having a diameter of one micron is shown resting on a substrate 10. FIG. 2 further illustrates a particle 12 having a diameter of approximately 0.3 micron representing the grain size of a fine automotive finish or paint. A smaller particle 13 further illustrates the smallest grain size comprising a black/white photographic film having a 0.2 micron diameter. By comparison, a particle 14 having a diameter of one nanometer is represented by an extremely small dot (particle) 14 in the center of the drawing. To improve the visibility of the scale, the nanoscale particles 16 are enlarged one hundred times. A particle or grain at or below 100 nanometers is generally understood to be within the nanoscale. Surface features at the nanoscale are appreciated to be very, very small.

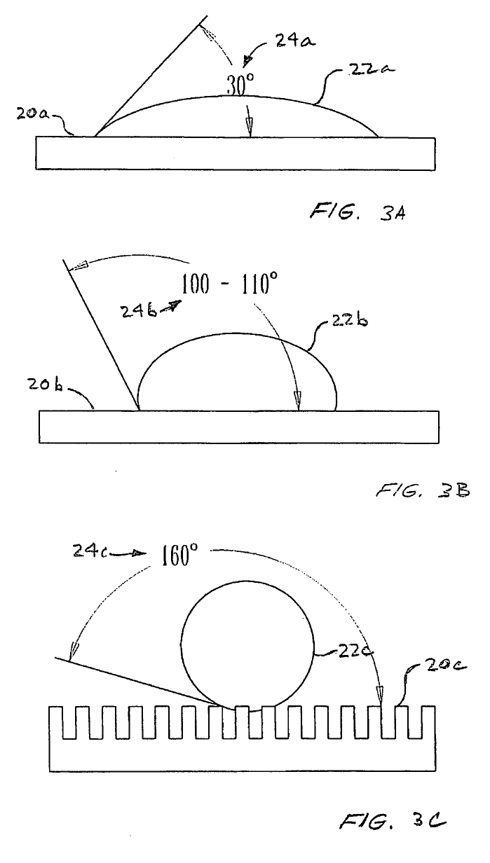

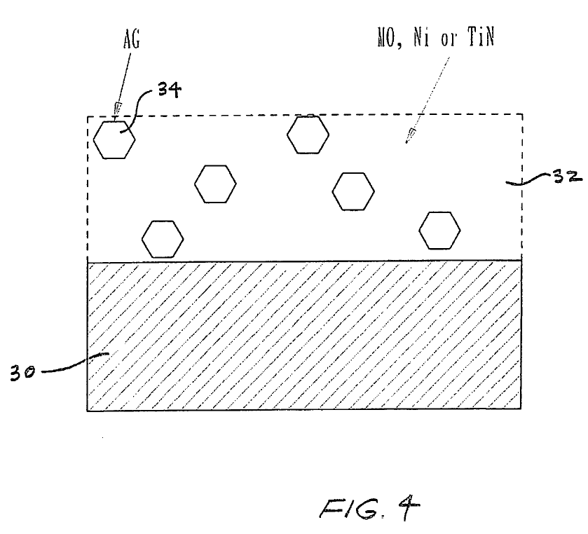

Materials that have been treated, modified or coated so as to have a nanostructure surface may appear smooth to the naked eye. However, under a powerful microscope, the surface appears to be rough and bumpy. The nanostructured surface comprises of discrete particles in a highly organized pattern. Each nanoparticle exhibits individual properties and contributes to a collective structure in a way that makes the surface controllable. Nanocrystalline powders deposited upon a material surface have been shown to increase hardness and other desirable properties. One desirable property is that of hydrophobicity. This property is illustrated in FIGS. 3A-3C where a water droplet is shown upon a surface. An untreated glass surface 20a is illustrated in FIG. 3A and is seen to have a small contact angle 24a. The water droplet 22a is seen to be spread over a large area in proportion to the contact angle 24a. In comparison, FIG. 3B illustrates a surface 20b coated with Teflon or PTFE. The contact angle 24b is greatly increased since the Teflon repels the water droplet 22b to some extent and is indeed hydrophobic. In another comparison, as illustrated in FIG. 3C, the surface 20c is treated with a nanostructure that is considered superhydrophobic such that the contact angle 24c is dramatically increased. There are quantum physical principles involved in this relationship between the nanosurface and the water droplet 22c. The nanostructure operates at the atomic level and the relationships at that level are dramatic.

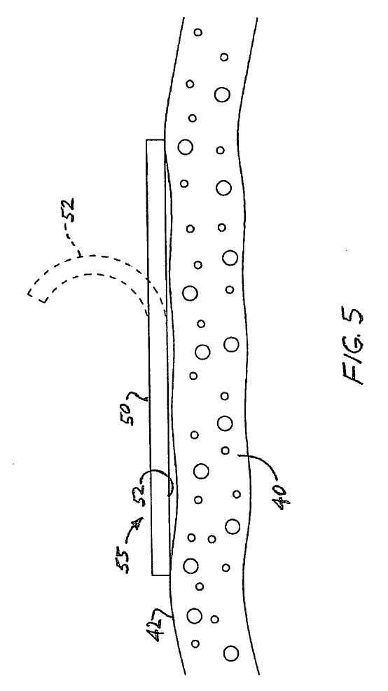

Nanoscale materials, characterized by grain sizes of less than 100 nm are demonstrating significant improvement in durability, flexibility and functional properties. In addition to being able to apply coatings made from nanophase powders, techniques themselves are being developed in which the processing parameters involved in the spraying actually produce the nanocrystalline structure. This has been achieved using a hypersonic plasma particle deposition (HPPD) process to apply SiC coatings. The materials and processes for developing a nanocoating are widely available. Nanopowders are produced in relatively large quantities and in a wide range of material. For instance, a nanocoating may be produced from metals or elements of the fourth major group of the periodic system or compounds of these elements. The processes for producing a nanocoated surface include direct deposition of materials upon a surface and subsequent curing and hardening of the material. A magnetron sputter technique is one mode of producing a nanocoating. This technique involves application in a vacuum. A solid base is coated with metallic or non-metallic layers. The coating material on the cathodes is atomized or sputtered by bombardment of the material with gas ions in the gas atmosphere. FIG. 4 illustrates a surface 30 that is coated with a nanostructure such as MO, Ni, or TiNi including AG 34.

Referring to FIG. 5, there is shown a portion of living tissue 40 having a surface 42 being in contact with an instrument 55. The instrument 55 has a nanocoat 52 providing a hydrophobic or hydrophilic surface that adheres well to tissue 40 temporarily and is easily removed by peeling. This property is achieved by the formation of vertical nanostructures or nanotubes that have sticking abilities stemming from 200 nm wide vertical structures. Capillary forces cause nanostructures with that diameter to stick to films of water or wet surfaces. Equally strong van der Waals forces enable them to attach to dry surfaces as well. Each vertical structure exerts only 10⁻⁷ N of force, but they are densely packed enough to collectively have an adhesive force of 10 N/cm², enough to suspend a 100-kg mass from a 10 cm² patch.

This property of nanocoatings makes them very useful for retractors, clips and other devices that contact tissue in a retentive or tractive manner. Nanostructure surfaces provide further bonding through extreme van der Waals interactions where there is no chemical interaction between the surfaces. These are intermolecular electromagnetic attractions between one molecule and a neighbouring molecule. All molecules experience intermolecular attractions, although in some cases those attractions are very weak. In another aspect of the invention, the extremely hydrophobic property of the nanostructure surface treatment or coating of a reusable, sterilisable surgical instrument prevents attachment of micro-organisms. For instance, a reusable grasper, clip applier, scissors, dissector or laparoscope that had a nanostructure treatment or coating may be very easily and reliably cleaned between uses as the bacteria would find it difficult to grow on the nanostructure surfaces because of the superhydrophobicity of the nanostructure surfaces, i.e., micro-organisms cannot attach to a surface. Moreover, the nanotreated surfaces easily withstand the temperatures of a common autoclave because they are formed and cured at temperatures well above those of autoclave sterilization. In addition, the critical pivot or hinge points of the reusable instruments are well preserved in the presence of nanocoatings and require little or no lubrication.

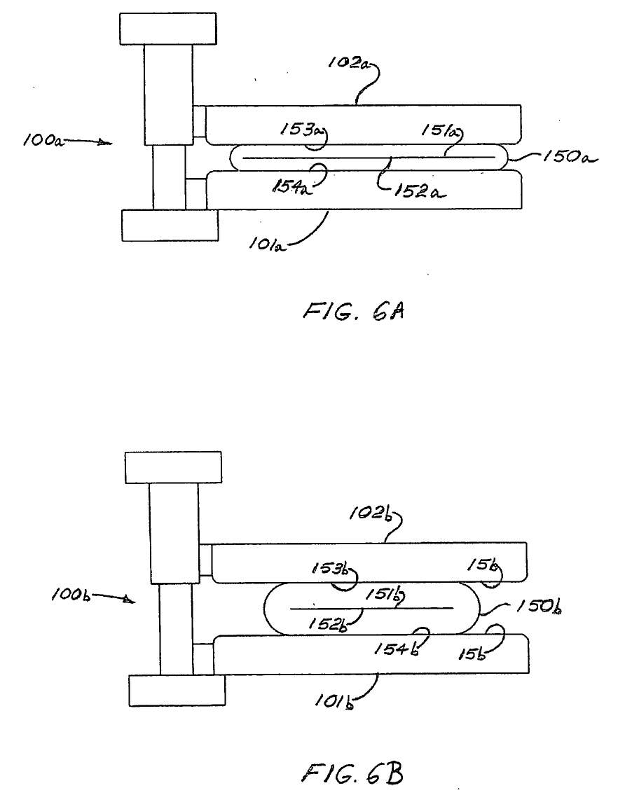

Referring to FIG. 6A, there is shown a surgical clip 100a commonly used to occlude blood vessels. The clip 100a has no coating on the contact portions 153a, 154a. The compression upon the vessel 150a must be excessive in order to maintain position upon the vessel. If the clip 100a slips off the vessel, it can represent a significant danger. The contact surfaces 153a, 154a of some surgical clips are serrated or covered with a fabric or the like to prevent slippage. Referring now to FIG. 6B, a clip 100b according to the present invention comprises a first jaw 101b, a second jaw 102b, and contact surfaces 153b, 154b operably attached on jaws 101b, 102b, respectively. The contact surfaces 153b, 154b may be modified, coated or treated with nanostructure materials 15b to provide enhanced traction without having to over-compress the tissue. For instance, a tissue contacting surface having a plurality of vertical nanotubes or nanohairs attaches to a smooth wet or dry surface by capillary attraction. As explained above, capillary forces cause nanostructures to stick to films of water or wet surfaces. More specifically, each vertical structure exerts only 10⁻⁷ N of force, but they are densely packed enough to collectively have an adhesive force of 10 N/cm², enough to suspend a 100-kg mass from a 10 cm² patch. The nanostructure may be vapor deposited and subsequently cured upon the tissue contacting portions of the clip.

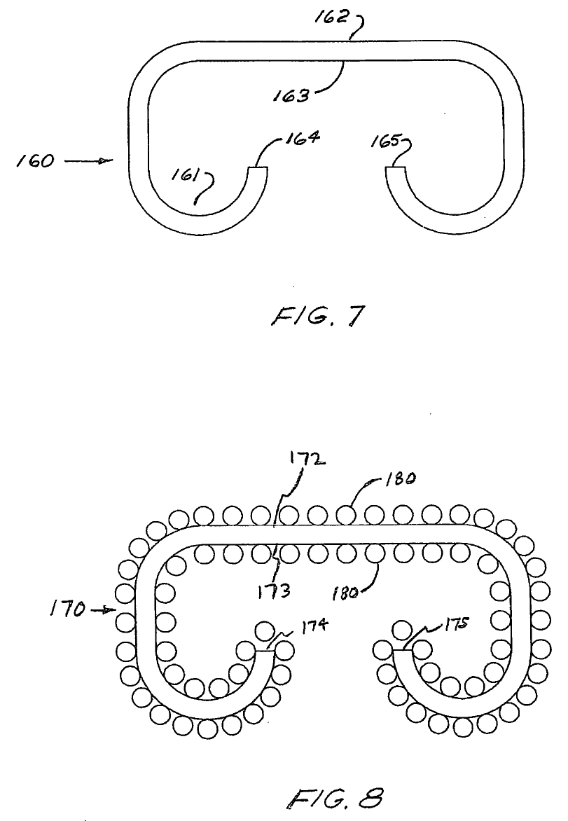

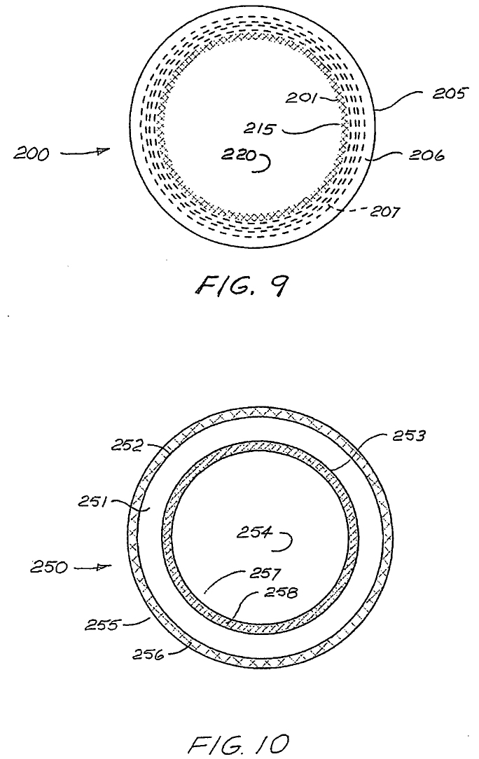

Referring to FIG. 7, there is shown a surgical staple 160 having surfaces 162, 163 that contact tissue, and portions 164, 165 that engage tissue. It should be noted that staples according to the prior art must be highly compressed in order for them to maintain position and function. Over compression of surgical staples is not desirable because it can adversely affect capillary blood flow and may result in tissue necrosis. FIG. 8 illustrates a surgical staple 170 in accordance with the invention having surfaces 172, 173 and ends 174, 175 treated, modified or coated with nanostructure material 180 that provides a hydrophilic attraction to living tissue. The surgical staple 170 of the present invention will maintain position and function without over compression of tissue. The pressure induced hydrophilic nanocoating provides a superlative matrix for encapsulation of the staple 170. Staples are generally formed from lengths of wire that are cut and pre-formed to a desired staple configuration. A nanocoated staple may be dipped, sprayed or otherwise coated with a silica, titanium, silver or other metal or plastic and subsequently heated to evaporate the solvents and stabilized in the presence of a vacuum and electrical arc. Alternately, a pulsed laser ablation may be used for deposition of the nanocomposite coatings. In these nanocomposites, nanocrystals of a transition metal nitride are embedded within about one monolayer thin amorphous tissue which yields a high material hardness and crack resistance. The small scale of the self-organizing nanostructures is well suited for deposition on surgical staples since the self-organizing property of the nanodeposition is not challenged by the small size and complex shape of a finished staple.

Yet another aspect of the present invention is illustrated in FIG. 9 where a vessel graft or stent 201 is shown within a vessel 200. The graft or stent 201 is coated, treated or modified with nanostructure materials 215 such as nanocrystalline titanium or silver that has been deposited on the tissue contacting surfaces so that they are attracted to and assimilated by the interior vessel wall 206. Additionally, a metal stent may be made of wires that have a nanostructure of vertical nanotubes or hairs that provide hydrophobicity due to capillary pressure except where compressive pressure breaks such capillary repulsion. In the regions where capillary pressure has been exceeded, the tissue contacting portions are thoroughly wetted by naturally occurring body fluid. When the stent or graft 201 is made of a porous or woven material, such as individual fibres coated with nanomaterials, improved tissue ingrowth 207 is expected. In time, such a nanocoated graft or stent 201 will provide a nearly natural fluid pathway 220.

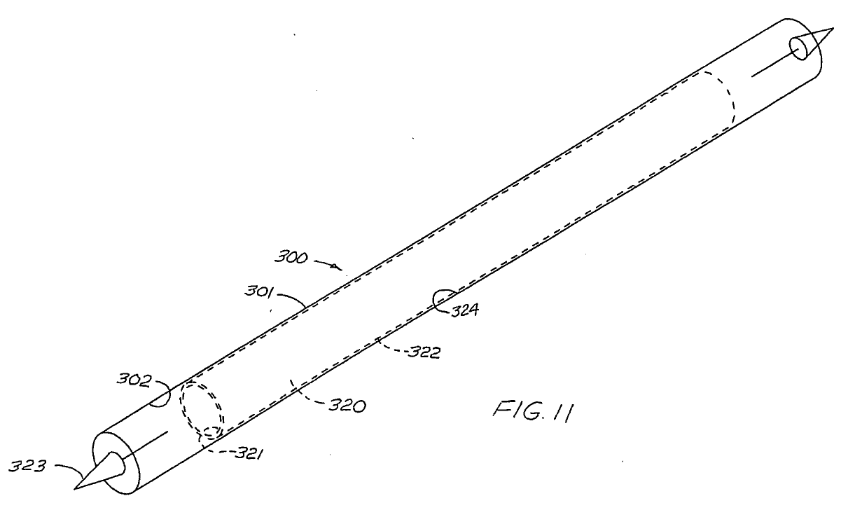

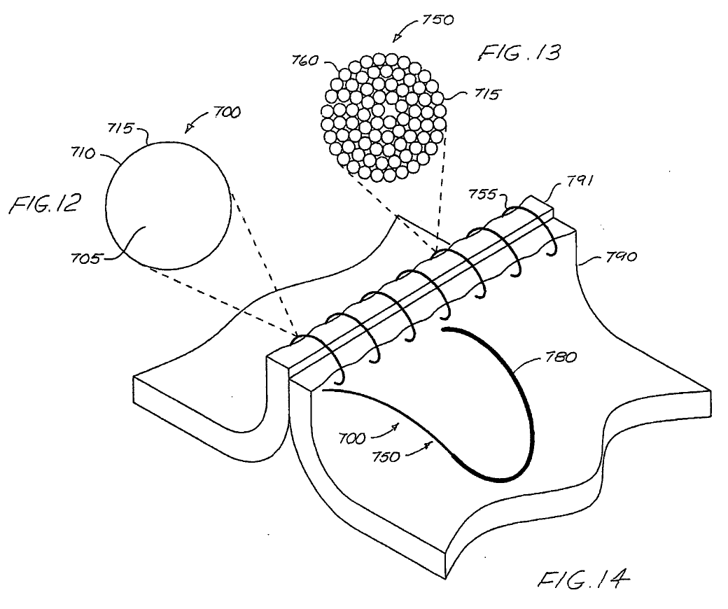

Referring now to FIG. 10, a length of medical tubing 250 is shown having an outer surface 255, an inner surface 257, a lumen 254 and a length. An embodiment of the present invention contemplates the use of nanostructure materials 256, 258 to modify, treat or coat the surfaces 255, 257. A first nanocoating may be applied to the outer surface 255 so as to provide a first effect and a second nanocoating may be applied to the inner surface 257 so as to provide a second effect. For instance, a hydrophobic nanocoating that comprises a plurality of self-organized nanostructures that respond to excessive capillary pressure by thoroughly wetting the treated surface may be applied to the exterior surface 255 so that the tube may be easily placed into a body passage and a similar hydrophobic nanocoating may be applied to the interior surface 257 so that normally sticky fluid borne components cannot collect and block the lumen 254 of the tube. It should be noted that a similar hydrophobic coating may be used on the exterior and the interior surfaces since it is the excessive compressive upon the exterior that renders the nanocoating hydrophilic. In another aspect, a nanocoating of TiO₂ (Titanium Dioxide) may be applied to the exterior of the tubing. Titanium dioxide is superhydrophilic and attracts water rather than repelling it. As further illustrated in FIG. 11, a stent or graft 320 that is treated or coated with a nanostructure may be placed into a vessel or body passage due to a hydrophilic external coating 322. The external coating 322 of the graft or stent is attracted to an intimal layer 324 of the vessel 300 and may be subsequently incorporated therein. Additionally, the luminal surface 321 may be treated or coated with a hydrophobic nanomaterial that provides conditions for flow maintenance.

Referring to FIGS. 12-14, it is appreciated that nanocoatings may be applied to individual fibers of a woven or braided stent, tube or graft in a front and back configuration so that the individual fibers exhibit the appropriate hydrophilic or hydrophobic properties. In addition, it must be noted that coating fibers with nanomaterials changes the dimension very little. Therefore, it finds great value in treating individual fibers 760 and suture 700, 750. For instance, monofilament suture 700 that is nanotreated 715 will take on smoother surface 710 characteristics that have been very difficult to achieve. Suture that is intended to be removed after a time may be coated, modified or treated with hydrophobic nanomaterials so that it will not attach to body tissue 790, 791 as healing occurs. Removal of a nanotreated suture will require less tension and will result in less pain and damage to healing tissue because naturally occurring fluids are not able to integrate with the surface due to the high water droplet contact angle of the superhydrophobic nanocoating. In addition, the superhydrophobic nature of nanocrystalline structures will prevent attachment of microbes and bacteria, and will therefore aid in the prevention of wound infection. This is an especially valuable aspect of the present invention when stranded suture 750 is used. The hydrophobic nature of nanocoatings will prevent the “wicking” of fluid that normally occurs when fluid conduits are sutured with stranded suture 750. Stranded suture 750 is not normally used in the anastomosis of bowel or colon because contaminate may leak through the suture 750 itself. A hydrophobic nanostructure coating or surface modification to the fibre 760 of a stranded suture 750 makes it waterproof. The extremely small size and self-organizing properties of nano particles and subsequent water-repellent structures provide repulsive forces that prevent the passage of a molecule of fluid through the interstices or weave-voids of a stranded suture. Only a nano-sized coating could possibly achieve the hydrophobicity required, bearing in mind that the individual strands of a stranded suture are measured in microns. Thicker coatings or waxes would add excessive dimension to the suture strands-strands.

In the case of monofilament sutures 700 that are to remain in place permanently, a hydrophilic nanocoating provides a nearly perfect matrix for encapsulation and incorporation of a suture. The process of encapsulation is greatly expedited by the application of hydrophilic nanocoatings to suture. An alternate embodiment of the present invention also contemplates the use of hydrophilic nanotreatment of stranded suture 750 where it is intended for permanent placement. The application of nanotechnology to extremely fine suture is also very important. Very fine suture, such as that used in ocular-surgery, neuro-surgery, cardiac and vascular surgery are greatly benefited by nanocoatings that add properties without adding significant dimension. Wire sutures used in orthopaedic surgery are greatly benefited by the properties of nanocoatings. They are more easily passed through tissue and bone and they are not subjected to the chemical reactions concomitant with residence in a living body. In addition, needles 780 used to place sutures 700, 750 are contemplated as part of the present invention, where such a nanocoated needle 780 is provided with a hydrophilic nanocoating so that it is easily passed through tissue 790, 791 without the normal drag associated with a bare-steel needle. This aspect of the present invention is especially valuable in the field of plastic or cosmetic surgery. Suture that is treated with nanostructure materials is rendered virtually non-reactive with tissue so that as healing occurs, the suture is not incorporated in the developing tissue. It may be possible to close cosmetic incisions with smaller gage suture as it is much more easily passed through tissue.

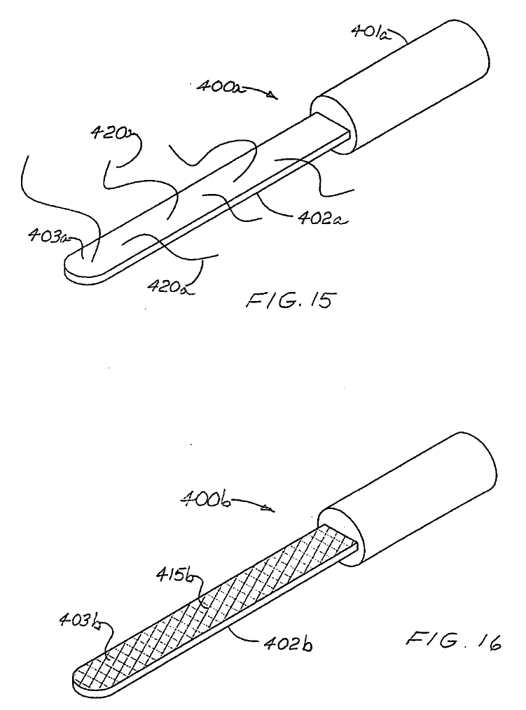

Referring to FIG. 15, there is shown an electrosurgical electrode 400a having a surface 403a that is either coated or uncoated with known materials such as PTFE. As tissue is heated and subsequently desiccated, proteins and collagen tends to adhere tenaciously to the surface 403a of the electrode 400a just as cooked meat sticks to a grill. As the eschar 420a builds up, the efficiency of the entire device deteriorates dramatically. In some cases the tissue sticks to the surface 403a to a degree that the device is no longer usable. FIG. 16 illustrates an electrosurgical device 400b in accordance with the invention comprising surfaces 402b, 403b that are modified, treated or coated with a nanostructure material 415b comprising, in one embodiment, titanium dioxide which is superhydrophilic and consequently self-cleaning. Generally, water droplets form on a ceramic at a contact angle of about 43 degrees which means that a ceramic electrosurgical instrument would be hydrophobic. Contamination of fluid droplets on the surface will occur over time as the electrosurgical device is used. However, fluid droplets on the surface of a photocatalytic, superhydrophilic ceramic will spread to form a contact angle of only 7 to 25 degrees. This means that surface wetting and rinsing is very uniform; fluids slide under and float away organic surface contaminates so that adhesion cannot occur. The density of the surface and the hydrophobicity defy adhesion of eschar 420. Electrosurgical devices include blades, probes, scissors, hooks, graspers and wires. All of these devices may be enhanced by the application of nanocoatings because a denser and more hydrophobic surface is provided.

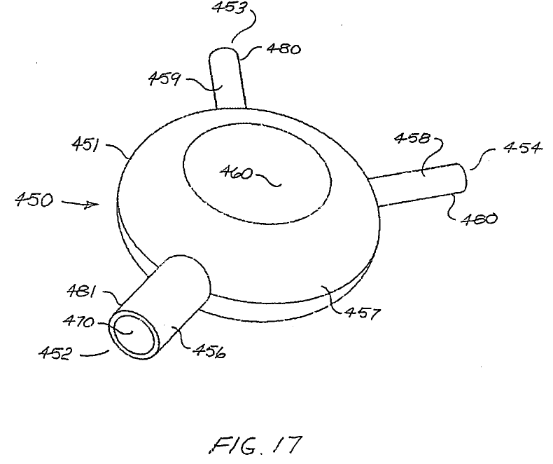

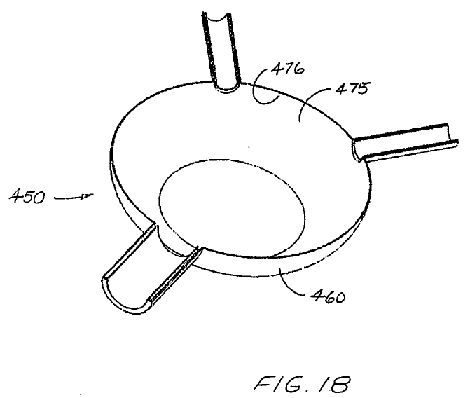

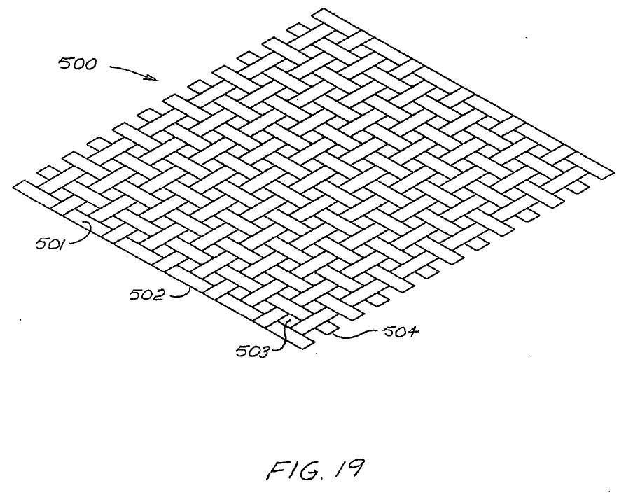

Referring to FIGS. 17 and 18, there is shown an artificial urinary bladder 450 having a hollow main body portion 460, an outlet portion 452, a first inlet portion 453 and a second inlet portion 454. The bladder 450 is sized and configured to replace a natural urinary bladder that has been removed due to disease. In one aspect, the artificial urinary bladder 450 comprises a flexible structure that is implanted permanently. There are two significant issues with this sort of implant. First, external adhesion of the bladder material to adjoining tissue is not desirable. Second, incrustation of the interior 475 of the bladder must be prevented. Nanocoating or modification of the bladder surfaces 457, 476 with nanomaterials according to the present invention provides a solution to both issues. First, the exterior 457 of the bladder may be coated with a specific hydrophobic material that allows flexibility without creating cracks in the surface 457. The external surface may be prepared, with a nanocoating comprising polypropylene that has been precipitated upon the bladder surface so that a fluid contact angle of about 160 degrees is maintained. This will prevent ingrowth of adjoining tissue and subsequent adhesion. The interior surface 476 of the bladder may be coated with specific hydrophobic nanomaterials and additional materials such as silver or titanium that resist encrustation and infection. In one embodiment, the artificial bladder 450 is fitted with fabric cuffs 480, 481 at the inlets 453, 454 and at the outlet 452. These cuffs 480, 481 are sized and configured to attach to the natural body conduits, i.e., ureters and urethra. The cuffs 480, 481 are preferably made of woven Dacron fabric. The present invention contemplates the permeation of the cuffs 480, 481 with hydrophilic nanostructure such as titanium dioxide that will promote tissue ingrowth and incorporation.

Referring now to FIG. 19, there is shown a portion of a woven or knitted fabric 500 having a plurality of crossing fibers 501, 504 and interstices 503. The fabric 500 is commonly used as a graft, sling, support or patch upon natural tissue. The fabric 500 is commonly attached to tissue by suture, staple, coil or glue. The present invention contemplates the coating of the fabric 500 on one or both sides with nanomaterials. For instance, a hydrophilic nanostructure comprising titanium dioxide may be applied to the fibers 501, 504 or the entire fabric 500 in order to promote tissue ingrowth and minimize rejection. In addition, one side may be coated with a hydrophilic nanostructure such as titanium dioxide while the opposite side is coated with a hydrophobic nanostructure such as precipitated polypropylene. This construction will prevent adhesion of adjoining tissue such as abdominal tissue, peritoneum and the like. It is especially noteworthy that nanostructures are inherently flexible and durable and therefore are well suited for fabric application.

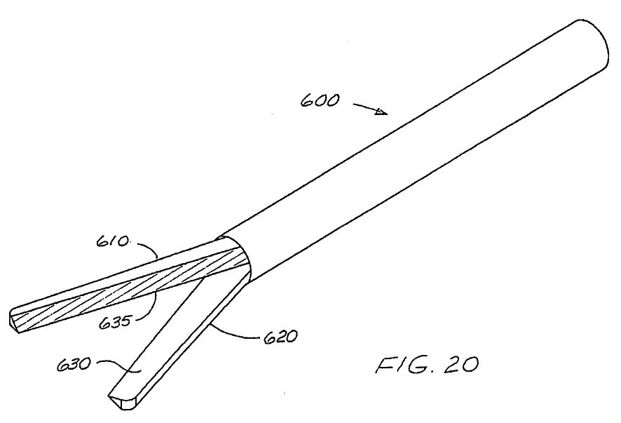

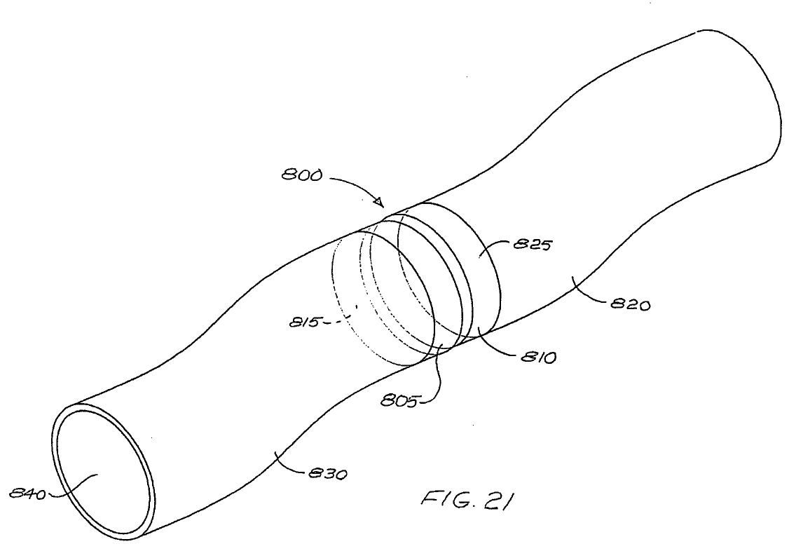

FIG. 20 illustrates a surgical scissor 600 having a pair of opposed blades 610, 620 and surfaces 635, 630, respectively. These blades 610, 620 must be very sharp and must be held in close contact with each other. This obviously presents mechanical issues such as friction and “break-away” motions. Lubrication of scissor blades is common to aid with the mechanical issues. However, lubrication soon breaks down in a surgical procedure and may become ineffective. These issues have also been addressed using thick coatings of PTFE or the like. These have been moderately successful. Scissor blades 610, 620 that are nanotreated according to the present invention do not require lubrication since the surfaces 635, 630 may be provided with a friction reducing wetness which derives from the superhydrophilic properties of titanium dioxide. In this case, moisture from tissue that is being cut forms water droplets with surface contact angles of 7 to 25 degrees. The tissue contacting surfaces are therefore thoroughly wetted. Alternately, the contacting surfaces may be treated with nanomaterials that are inherently repulsive or non-reactive such as precipitated polypropylene. The nanocoatings are so thin that the integrity of the design is not compromised by the application of the coating to the opposing surfaces 630, 635.

A luminal anastomosis device 800 is shown in FIG. 21 having a first attachment portion 805 and a second attachment portion 810. Such devices would normally be attached to residual lumens 820, 830 with suture during a resection procedure. The sutures would attach the tissue 820, 830 to the attachment portions 805, 810. It is appreciated that there is the potential for leaks between the suture passes. A unique property of specific hydrophilic nanocoatings is that of tissue adhesion. As such, the present invention contemplates the use of nanostructures to attach living tissue 820, 830 to the attachment portions 805, 810 of the anastomosis device 800 without the use of suture. Various mechanical capturing and holding portions may be included where the nanocoatings provide attachment of an occlusion. An embodiment of the anastomosis device may provide a hydrophobic nanocoating 815 upon the inner surface of the connecting portions 805, 810 and a hydrophilic nanocoating upon the outer tissue-contacting surface 825 of the connecting portions 805, 810.

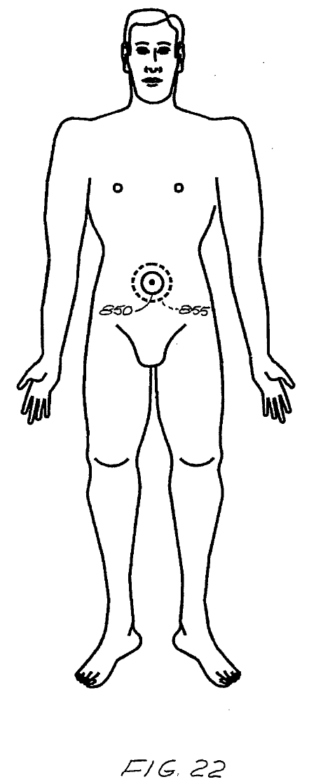

FIG. 22 illustrates an additional use for the present invention. A dialysis port 850 is illustrated. The port 850 is a permanent opening into the abdominal cavity of a living human for the regular introduction and removal of dialysis fluid. This is referred to as peritoneal dialysis. Peritoneal dialysis has an associated problematic issue. That is, infection of the port site 855. Treatment of the access port 850 with nanomaterials will prevent infection since micro-organisms cannot attach or grow on nanotreated surfaces.

It will be understood that many other modifications can be made to the various disclosed embodiments without departing from the spirit and scope of the invention. For at least these reasons, the above description should not be construed as limiting the invention, but should be interpreted as merely exemplary of preferred embodiments.

1. A nanostructure surface treatment for a medical device having an external surface, wherein the treatment is applied on the external surface to provide a hydrophobic or a hydrophilic surface.

2. The nanostructure surface treatment of claim 1, wherein the treatment comprises titanium dioxide.

3. The nanostructure surface treatment of claim 1, wherein the treatment comprises tungsten-carbide-cobalt.

4. The nanostructure surface treatment of claim 1, wherein the treated surface of the device has contact angles greater than or equal to 150 degrees.

5. The nanostructure surface treatment of claim 1, wherein the device is a clip, a staple, a retractor, a suture, a manipulator, a grasper, a clip-applier, a scissors, a dissector, an electrosurgical device, or a laparoscope.

6. The nanostructure surface treatment of claim 5, wherein the treatment further facilitates at least one of traction, retention, and occlusion.

7. The nanostructure surface treatment of claim 1, wherein the treated surface includes nanoscopic structures having nearly vertical sidewalls.

8. The nanostructure surface treatment of claim 7, wherein the vertical sidewalls provide a negative capillary effect.

9. The nanostructure surface treatment of claim 1, wherein the treated surface includes nanoscopic structures providing the external surface with a high-contact angle and a low sliding resistance on the surface.

10. The nanostructure surface treatment of claim 7, wherein the vertical sidewalls have a width of about 200 nm.

11. The nanostructure surface treatment of claim 8, wherein the vertical sidewalls attach to a wet surface by the negative capillary effect.

12. The nanostructure surface treatment of claim 11, wherein the van der Waals forces of the vertical sidewalls enable the treated surface to attach to a dry surface.

13. The nanostructure surface treatment of claim 1, wherein the treatment is vapor deposited and cured on the device.

14. The nanostructure surface treatment of claim 1, wherein the treatment is laser blasted on the device.

15. The nanostructure surface treatment of claim 13 or 14, wherein the treated device is dipped, sprayed or coated with at least one of silica, titanium, silver or other metal or plastic and subsequently heated to evaporate the solvents and stabilize in the presence of a vacuum.

16. The nanostructure surface treatment of claim 1, wherein the device further comprises a lumen having an internal surface.

17. The nanostructure surface treatment of claim 16, wherein the internal surface is coated, treated, or modified with a nanostructure including fluoroalkylsilane, nanocrystalline titanium, or silver.

18. The nanostructure surface treatment of claim 16, wherein the internal surface is treated through at least one of hydrolysis, condensation reactions, screen printing, electrostatic glazing, and spraying.

19. The nanostructure surface treatment of claim 16, where in the device is an access tube, a stent, a graft, a medical tubing, or a valve.

20. An artificial medical device, comprising:
a hollow body portion having an internal surface and an external surface;
an inlet portion operably attached to the body portion; and
an outlet portion operably attached to the body portion,
wherein the external surface of the body portion is coated, treated, or modified with a hydrophobic nanostructure surface treatment, and the internal surface of the body portion is coated, treated, or modified with a hydrophilic nanostructure surface treatment.

21. The artificial medical device of claim 20, wherein the device is an artificial bladder.

22. The artificial medical device of claim 20, wherein the device is a dialysis port.

23. The artificial medical device of claim 20, wherein the hydrophobic nanostructure surface treatment comprises titanium dioxide.

24. The artificial medical device of claim 23, wherein the hydrophobic nanostructure surface treatment further comprises polypropylene.

25. The artificial medical device of claim 20, wherein the hydrophilic nanostructure surface treatment comprises fluoroalkylsilane, nanocrystalline titanium, or silver.

26. The artificial medical device of claim 20, wherein the internal surface is treated through at least one of hydrolysis, condensation reactions, screen printing, electrostatic glazing, and spraying.

27. The artificial medical device of claim 20, further comprising a cuff for at least at one of the inlet portion and the outlet portion to attach to a body conduit.

28. The artificial medical device of claim 27, wherein the cuff is made of fabric and is permeated with a hydrophilic nanostructure surface treatment.

29. A surgical fabric comprising a plurality of crossing fibers, a plurality of interstices, and two surfaces, wherein at least one of the two surfaces is coated, treated, or modified with a hydrophilic or a hydrophobic nanostructure surface treatment.

30. The surgical fabric of claim 29, wherein the other of the two surfaces is coated, treated, or modified with a hydrophobic or a hydrophilic surface treatment different from the first surface treatment.

31. The surgical fabric of claim 29 or claim 30, wherein the hydrophilic nanostructure treatment comprises titanium dioxide.

32. The surgical fabric of claim 29 or claim 30, wherein the hydrophobic nanostructure treatment comprises precipitated polypropylene.
